(19) United States
(12) Patent Application Publication
(10) Pub. No.: US 2019/0015446 A1
(43) Pub. Date: Jan. 17, 2019

(54) MEDICINAL COMPOSITION FOR IMPROVING PROGNOSIS AFTER RESTART OF PATIENT'S OWN HEARTBEAT

(71) Applicants: KEIO UNIVERSITY, Tokyo (JP); TAIYO NIPPON SANSO CORPORATION, Tokyo (JP)

(72) Inventors: Masaru Suzuki, Tokyo (JP); Kei Hayashida, Tokyo (JP); Tomoyoshi Tamura, Tokyo (JP); Motoaki Sano, Tokyo (JP); Yoshiki Nishiwaki, Tokyo (JP)

(21) Appl. No.: 16/066,009

(22) PCT Filed: Dec. 21, 2016
(86) PCT No.: PCT/JP2016/088172

§ 371 Date: Jun. 25, 2018

(30) Foreign Application Priority Data
Dec. 28, 2015 (JP) 2015-257318

(51) Int. Cl. A61K 33/00; A61P 9/10; A61P 25/28

(52) U.S. Cl. CPC A61K 33/00; A61P 25/28; A61P 9/10

(57) ABSTRACT

[Problem] To provide a medicinal composition that is capable of improving the prognosis after the restart of the patient's own heartbeat even after the passage of a considerably long period of time after the restart of the patient's own heartbeat. [Solution] A gaseous medicinal composition for improving the prognosis alter the restart of the patients own heartbeat, said medicinal composition being to be administered to a human subject after the passage of at least 30 minutes after the restart of the patient's own heartbeat following cardiorespiratory arrest for at least 10 minutes. The medicinal composition according to the present invention, which contains hydrogen gas, is characterized in that a group of human patients administered with the medicinal composition shows improved prognosis after the restart of the patient's own heartbeat compared with another group of human patients not administered with the medicinal composition.

### TECHNICAL FIELD

The present invention relates to a pharmaceutical composition for improving prognosis after return of spontaneous circulation following cardiopulmonary arrest.

### BACKGROUND ART

Patients resuscitated from out-of-hospital cardiac arrest, even if their lives can be saved, have severe aftereffects in the brain or the heart, a low rehabilitation rate, and very poor vital prognosis. According to the multicenter observational study of out-of-hospital cardiac arrest patients conducted in 2012 (58 hospitals in Kanto Area in Japan), 920 resuscitated patients hospitalized after return of spontaneous circulation exhibited a 1-month survival rate of 21.6% and a 1-month consciousness recovery rate of 13.3%. Therapeutic hypothermia, only one treatment found to have efficacy on the organ protection of resuscitated patients, requires a large-scale apparatus and the involvement of many healthcare professionals in charge thereof and is therefore practiced only in some facilities such as university hospitals. Thus, the development of a novel treatment method in place of the therapeutic hypothermia is urgently necessary for improvement of prognosis of resuscitated patients.

Organ damage in the brain, the heart, or the like after cardiac arrest resuscitation is also called post-cardiac arrest syndrome (PCAS) and is considered to involve reactive oxygen species or free radicals generated after return of spontaneous circulation. Hydrogen gas is a selective radical scavenger that eliminates only a hydroxy radical which is a harmful reactive oxygen species, and has a very characteristic property of being able to pass through a cell membrane and reach organelle, irrespective of blood flow, because of its small molecular weight and excellent diffusing power (Non Patent Literature 1). Also, the inhalation of hydrogen gas (49%) used in the prevention of decompression sickness in humans and known to manifest no adverse reaction even if healthy persons inhale the hydrogen gas (Non Patent Literature 2).

Against this backdrop, the present inventors have found that the inhalation of hydrogen working as a selective radical scavenger can improve brain and heart functions and vital prognosis after cardiopulmonary arrest resuscitation in rats (Non Patent Literatures 3 and 4).

### CITATION LIST

### Non Patent Literature

[Non Patent Literature 1] Nat Med. 2007 June; 13 (6): 688-94

[Non Patent Literature 2] Journal of Applied Physiology Mar. 1, 1994 vol. 76 No. 3 1113-1118

[Non Patent Literature 3] Hayashida K, Sano M, Hori S et al., Journal of the American Heart Association 2012; doi: 10.1161/JAHA.112.003459

[Non Patent Literature 4] Circulation. 2014 Dec. 9; 130 (24): 2173-80

### SUMMARY OF INVENTION

### Technical Problem

The experimental results obtained in rats suggest that hydrogen inhaled from before return of spontaneous circulation to immediately after the return of spontaneous circulation functioned favorably as a selective radical scavenger and reduced damage ascribable to reactive oxygen species after the return of spontaneous circulation. On the other hand, its safety or efficacy in humans is still unknown.

At present, the administration of hydrogen gas to humans requires the consent of patients themselves or their relatives or other persons concerned. Therefore, it is very difficult to carry out hydrogen inhalation in humans from before return of spontaneous circulation to immediately after the return of spontaneous circulation, as in the previous practice in rats. In other words, it is almost impossible to obtain the consent from patients themselves within a period from before return of spontaneous circulation to immediately after the return of spontaneous circulation. Furthermore, a certain amount of time is generally required for obtaining the consent to hydrogen inhalation from persons related to the patients. The generation of reactive oxygen species at a reflow site of blood is considered to occur several minutes after return of spontaneous circulation (Circulation. (2002) 105: 2332-2336; and Proc. Natl. Acad. Sci. USA Vol. 86, pp. 4695-4699 (June 1989)). Hence, the hydrogen inhalation in humans is often started after a lapse of a considerable period of time from the generation of reactive oxygen species in the body. In such a case, effects similar to those previously confirmed in mice cannot be expected because organ damage ascribable to reactive oxygen species has already progressed.

Accordingly, an object of the present invention is to provide a pharmaceutical composition that can improve prognosis after return of spontaneous circulation even after a lapse of a considerable period of time from the return of spontaneous circulation.

### Solution to Problem

In the course of studying the safety and efficacy of hydrogen inhalation in patients resuscitated from out-of-hospital cardiac arrest, the present inventors have found that, totally unexpectedly, hydrogen inhalation can significantly improve prognosis after return of spontaneous circulation even is human patients after a lapse of a considerable period of time from the return of circulation following cardiopulmonary arrest. As already mentioned, the effect of hydrogen gas as a selective radical scavenger cannot explain such successful improvement of prognosis after return of spontaneous circulation even in patients in which organ damage ascribable to reactive oxygen species has already progressed. Thus, the findings gained by the present inventors are surprising.

The present invention is based on the findings described above and encompasses the following features:

[1] A gaseous pharmaceutical composition for improving prognosis after return of spontaneous circulation in a human subject after a lapse of 30 minutes or longer from the return of spontaneous circulation following cardiopulmonary arrest for at least 10 minutes,

the pharmaceutical composition comprising hydrogen gas, wherein

the prognosis after return of spontaneous circulation is improved in a human patient group with the administration of the pharmaceutical composition as compared with a human patient group without the administration of the pharmaceutical composition.

[2] The pharmaceutical composition according to [1], wherein

the improvement of the prognosis is improvement of brain function.

[3] The pharmaceutical composition according to [1] or [2], wherein

the human subject is a subject having a Glasgow Coma Scale (GCS) score of 8 or lower.

[4] The pharmaceutical composition according to any of [1] to [3], wherein

the “improvement of prognosis after return of spontaneous circulation” is improvement at at least 90 days after the return of spontaneous circulation.

[5] The pharmaceutical composition according to any of [1] to [4], wherein

the “improvement of prognosis after return of spontaneous circulation” is determined on the basis of a Cerebral Performance Category (CPC) score.

[6] The pharmaceutical composition according to any of [1] to [5], wherein

the pharmaceutical composition is continuously administered for at least 18 hours from the start of administration.

[7] The pharmaceutical composition according to any of [1] to [6], wherein

the pharmaceutical composition further comprises oxygen gas.

[8] The pharmaceutical composition according to any of [1] to [7], wherein

the pharmaceutical composition further comprises an inert gas.

[9] The pharmaceutical composition according to any of [1] to [8], wherein

the hydrogen concentration in the pharmaceutical composition is 0.1% to 4.0%.

[10] The pharmaceutical composition according to [9], wherein

the hydrogen concentration in the pharmaceutical composition is 1.0% to 2.0%.

[11] The pharmaceutical composition according to any of [1] to [10], wherein

the cardiopulmonary arrest is caused by acute myocardial infarction, cardiomyopathy or hyperkalemia.

One of or any combination of two or more of the aspects of the present invention described above is also included in the scope of the present invention.

### Advantageous Effects of Invention

The present invention provides a pharmaceutical composition that can improve prognosis after return of spontaneous circulation even after a lapse of a considerable period of time from the return of spontaneous circulation.

### DESCRIPTION OF EMBODIMENTS

Hereinafter, embodiments of the present invention will be specifically described.

The present invention relates to a pharmaceutical composition for improving prognosis after return of spontaneous circulation. The pharmaceutical composition of the present invention can be effective for as arbitrary human patient who has experienced return of spontaneous circulation following cardiopulmonary arrest. Meanwhile, the present inventors have newly found that hydrogen gas can improve prognosis after return of spontaneous circulation even in human patients who have experienced organ damage ascribable to free radicals, after a lapse of a considerable period of time from the return of spontaneous circulation following cardiopulmonary arrest. Therefore, the pharmaceutical composition of the present invention can be administered, particularly, to a human patient as a recipient after a lapse of a given cardiopulmonary arrest duration and a considerable period of time from subsequent return of spontaneous circulation.

Thus, the pharmaceutical composition of the present invention can be administered to a human patient, as a recipient, who has experienced cardiopulmonary arrest over a time long enough to generate injurious reactive oxygen species or free radicals due to return of spontaneous circulation following the cardiopulmonary arrest. In the present invention, such a time is typically at least 10 minutes. On the other hand, the upper limit of the cardiopulmonary arrest duration is not particularly limited as long as rehabilitation after resuscitation is expected. At present, 35 minutes from cardiopulmonary arrest are used as a guide for terminating resuscitation.

The pharmaceutical composition of the present invention can also be administered to a human patient as a recipient after a lapse of a time long enough to generate injurious reactive oxygen species or free radicals after return of spontaneous circulation following cardiopulmonary arrest. Such a time can be at least 30 minutes, preferably at least 1 hour, at least 2 hours, or at least 3 hours. On the other hand, the upper limit of the time from after the return of spontaneous circulation to the start of administration is not particularly limited. As described below in Examples, the present inventors have suggested, for the first time, that hydrogen gas when administered to human patients after return of spontaneous circulation does not always exert an effect only as a selective radical scavenger. Thus, the pharmaceutical composition of the present invention may be administered even after the end of production of reactive oxygen species or free radicals after return of spontaneous circulation.

In the present invention, the cause of the cardiopulmonary arrest is not particularly limited and may be a cardiogenic disease or may be a non-cardiogenic disease. In the present invention, examples of the cardiogenic disease can include, but are not limited to, acute coronary syndromes (ST-elevation myocardial infarction, non-ST-elevation myocardial infarction, unstable angina pectoris, etc.), cardiomyopathy (hypertrophic, dilated, restrictive, arrhythmogenic right ventricular, or unclassified cardiomyopathy), myocarditis, inherited or acquired arrhythmic diseases (long QT syndrome, Brugada syndrome, etc.), preexcitation syndrome, and sick sinus syndrome.

In the present invention, examples of the non-cardiogenic disease can include, but are not limited to, aorta dissection, cardiac tamponade, pulmonary embolism, sepsis, acidosis, hypoxemia, hypercapnia, suffocation, near-drowning, stroke, trauma, massive hemorrhage, tension pneumothorax, hypothermia, hypoglycemia, poisoning (tricyclic antidepressants, opioids, beta blockers, calcium receptor antagonists, digitalis, parasympathomimetic agents, adenosine, adenosine triphosphate, etc.), and electrolyte abnormality (hyperkalemia, etc).

In the present invention, the term “improvement of prognosis” refers to improvement of an arbitrary condition necessary for a patient to be rehabilitated after cardiopulmonary resuscitation. Examples of such a condition include, but are not limited to, survival rates, brain function, and heart function. In this respect, the phrase “prognosis after return of spontaneous circulation is improved as compared with a human patient group without the administration of the pharmaceutical composition” includes improvement of the degree of the arbitrary condition at a certain point in time after the return of spontaneous circulation as compared with the human patient group without the administration of the pharmaceutical composition of the present invention, and shortening of a time to achieve a given level of improvement in the arbitrary condition as compared with the human patient group without the administration of the pharmaceutical composition of the present invention.

In one embodiment of the present invention, the “improvement of prognosis” is improvement of brain function. The brain function can typically be determined on the basis of a Cerebral Performance Category (CPC) score. The CPC score is an index well known to those skilled in the art, and each score is as described in Table 1 below.

TABLE 1
Cerebral Performance Category (CPC)

(1) CPC 1: Good performance
Clear conscious, able to work and lead a normal life. May
have minor neurological or psychological deficits such as mild
dysarthria, minor cranial nerve disorders, or non-
incapacitating hemiparesis.
(2) CPC 2: Moderate disability
Conscious, sufficient cerebral function for part-time work in
sheltered environment or independent activities of daily life
such as dress, travel, or food preparation. May have
hemiplegia, seizures, ataxia, dysarthria, dysphagia, or
permanent memory or mental disability.
(3) CPC 3: Severe disability
Conscious, dependent on others for daily support because of
impaired brain function. Has at least limited cognition. A
wide range of cerebral abnormalities from severe memory
disturbances or dementia to patients who are paralyzed and can
communicate only with their eyes, as in the locked-in
syndrome.
(4) CPC 4: Coma
Unconscious, vegetative state, lack of cognition. Lack of
verbal or psychological interaction with environment.
(5) CPC 5: Death or brain death

The pharmaceutical composition of the present invention is characterized in that it is a gaseous pharmaceutical composition comprising hydrogen gas. The pharmaceutical composition of the present invention is also characterized in that it is continuously administered over a predetermined time to a human patient because of being a gaseous pharmaceutical composition. In the present invention, the hydrogen atom may be any of its all isotopes, i.e., protium (P or ¹H), deuterium (D or ²H), and tritium (T or ³H). Thus, P₂, PD, PT, DT, D₂ and T₂ may be included as molecular hydrogen. In a preferred aspect of the present invention, 99% or more of the hydrogen gas contained in the pharmaceutical composition of the present invention is natural molecular hydrogen P₂.

The pharmaceutical composition of the present invention can further comprise oxygen gas. The oxygen gas may be present in the form of a mixed gas by mixing with the hydrogen gas in advance, or may be mixed with the hydrogen gas immediately before or at the time of administration to a subject.

The pharmaceutical composition of the present invention can further comprise an inert gas. The inert gas is used for the purpose of preventing the explosion of the hydrogen gas or the oxygen gas and adjusting the concentration thereof and can thus be present in the form of a mixed gas with the hydrogen gas and/or the oxygen gas. Examples of the inert gas that can be used in the pharmaceutical composition of the present invention can include, but are not limited to, nitrogen gas, helium gas, and argon gas. In one embodiment of the present invention, inexpensive nitrogen gas is used as the inert gas.

In the pharmaceutical composition of the present invention, the concentration range of the hydrogen gas can be, but is not limited to, for example, any concentration from 0.1 to 4.0% (v/v). The lower limit value of the hydrogen gas concentration can be set as the lower limit value of a concentration at which the pharmaceutical composition can exert the effect of improving prognosis after return of spontaneous circulation. Thus, the minimum concentration that permits improvement of prognosis after return of spontaneous circulation can be appropriately set according to the severity of the patient, the cardiopulmonary arrest duration, the time from the return of spontaneous circulation to the start of administration, the type of the disease responsible for cardiac arrest, sex, age, etc. In one embodiment, the lower limit value of the hydrogen gas can be selected as any value from 0.1 to 1.0%, for example, 0.5%. On the other hand, the upper limit value of the hydrogen gas concentration is set from the viewpoint of safety because the lower explosion limit of hydrogen in air is 4%. Thus, the upper limit value of the hydrogen gas can be selected as any concentration of 4% or lower, for example, 3.0%, 2.5% or 2.0% as long as safety is ensured.

In the pharmaceutical composition of the present invention, the concentration of the oxygen gas can be in the range of 21% to 99.9% (v/v) on the precondition that the hydrogen gas concentration is 0.1 to 4.0% (v/v).

In the pharmaceutical composition of the present invention, the concentration of the inert gas is set in a range in which the concentration of the hydrogen gas and/or the oxygen gas is properly maintained, and the explosion-proof effects of these gases are obtained. Thus, those skilled in the art can appropriately set the concentration of the inert gas to a proper concentration according to the concentration of the hydrogen gas and/or the oxygen gas used. When the inert gas is, for example, nitrogen gas, such a concentration of the inert gas can be arbitrarily adopted in the range of, for example, 0 to 78.9% (v/v).

The concentration of each gas used throughout the present specification is indicated by a content at 20° C. at 101.3 kPa.

The pharmaceutical composition of the present invention may further comprise other gases such as carbon dioxide in the atmosphere, air, or anesthesia gas, etc. without impairing the effect of the present invention brought about by the hydrogen gas.

The pharmaceutical composition of the present invention can be administered to the subject, for example, by inhalation using an inhalation means. Examples of such an inhalation means can include, but are not limited to, inhaling masks. Preferably, the inhaling mask covers the mouth and the nose of the subject at the same time so as to achieve administration at a proper concentration to the subject.

In one embodiment of the present invention, the pharmaceutical composition of the present invention is provided in a form that can be administered directly to the subject. As one example, in this embodiment, the pharmaceutical composition of the present invention is provided in the form of a mixed gas prepared by mixing in advance the hydrogen gas and the inert gas, the oxygen gas for respiration, and other arbitrary gasses at proper concentrations.

In another aspect of the present invention, the pharmaceutical composition of the present invention is provided in a form that is prepared immediately before or at the time of administration to the subject. As one example, in this embodiment, the pharmaceutical composition of the present invention is provided by connecting a container containing a mixed gas of the hydrogen gas and the inert gas and a container containing the oxygen gas to an inhaling mask via piping, and sending these gases to the patient at a flow rate that attains their concentrations proper for administration to the subject. In one aspect of the present invention, the container may be in the form of a portable gas cylinder as well as, for example, a large storage tank installed outdoors. The gas may be contained in the form of a compressed gas in the container or may be contained in a liquefied form in a liquid gas container (LGC). As another example, in the present invention, the hydrogen gas, the oxygen gas, and the inert gas such as nitrogen gas may each be supplied from a gas generation apparatus. Examples of such a generation apparatus include, but are not limited to, oxygen concentrator for oxygen gas, and hydrogen generation apparatuses based on water electrolysis for hydrogen gas.

In an alternative aspect of the present invention, the pharmaceutical composition of the present invention is provided by supplying the hydrogen gas to a sealed chamber such that the concentration of the gas is kept constant. As one example, in this embodiment, the pharmaceutical composition of the present invention is provided by supplying, to a sealed chamber where the subject is present, a mixed gas of the hydrogen gas and the inert gas at a flow rate that keeps the hydrogen concentration at a proper concentration in the sealed chamber.

The pharmaceutical composition of the present invention can be provided in the form of one or more gas-containing containers or in the form of a device for gas inhalation comprising: a plurality of gas-containing containers; an inhalation means for gas inhalation; and piping which connects the plurality of containers to the gas inhalation means. In one embodiment of the present invention, preferably, the device for gas inhalation comprises a control mechanism which controls a gas flow rate to the gas inhalation means from each gas-containing container.

The administration period of the pharmaceutical composition of the present invention is not particularly limited as long as the period allows the pharmaceutical composition of the present invention to expert the effect of improving prognosis. Those skilled in the art can appropriately set the administration period to a proper period according to the severity of the patient, the cardiopulmonary arrest duration, the time from the return of spontaneous circulation to the start of administration, the type of the disease responsible for cardiac arrest, sex, age, etc. Such a period can be, but is not limited to, for example, at least 1 hour, at least 3 hours, at least 6 hours, at least 9 hours, at least 12 hours, at least 15 hours, at least 18 hours, at least 21 hours, at least 24 hours, at least 2 days, at least 5 days, at least 10 days, at least 15 days, at least 20 days, at least 25 days, or at least 30 days.

The pharmaceutical composition of the present invention is not limited by the number of doses and can be administered at a plurality of times. The dose interval and the number of doses of the pharmaceutical composition of the present invention can be appropriately set to a proper dose interval and number of doses according to the symptoms of the patient.

The pharmaceutical composition of the present invention may be used in combination with an additional effective treatment for improvement of prognosis after return of spontaneous circulation. Examples of such a treatment can include, but are not limited to, therapeutic hypothermia.

The pharmaceutical composition of the present invention can significantly improve prognosis after return of spontaneous circulation even in a human patient after a lapse of a considerable period of time from the return of spontaneous circulation following cardiopulmonary arrest. For example, as shown below in Examples, the pharmaceutical composition of the present invention improved brain function to attain CPC1 (good cerebral performance) at 90 days after the return of spontaneous circulation for all surviving human patients in a coma having a Glasgow Coma Scale (GCS; well known to those skilled in the art, as a 15-point scale for evaluating the severity of impaired consciousness) score of 8 or lower. This indicates that the pharmaceutical composition of the present invention can improve prognosis very favorably even in a human patient having a considerably low level of consciousness at the time of administration after return of spontaneous circulation.

The terms used in the present specification are given for illustrating particular embodiments and are not intended to limit the invention.

The term “comprising” used in the present specification means that described items (members, steps, factors, numbers, etc.) are present and the presence of the other items (members, steps, factors, numbers, etc.) is not excluded therefrom, unless the context evidently requires different interpretation.

It should be understood that the disclosures of the literatures cited herein are incorporated herein by reference in their entirety. Those skilled in the art can exploit the disclosed contents relating to these cited literatures as part of the present specification, without departing from the spirit and scope of the present invention, according to the context of the present specification

Hereinafter, the present invention will be more specifically described with reference to Examples. However, the present invention is not limited by Examples given below by any means.

### EXAMPLES

Hydrogen inhalation test on subject with post-cardiac arrest syndrome (PCAS)

1. Test Subject

In this test, the selected subjects were patients who satisfied particular criteria: the patients were each raced to a hospital due to out-of-hospital cardiac arrest, resuscitated by return of spontaneous circulation during the emergency transport or in an emergency room, particularly aged 20 to 80, and in a coma with a GCS score of 8 or lower in terms of the level of consciousness at the start of inhalation.

In this clinical trial, the patients after return of spontaneous circulation were in a coma, and it was impossible to obtain their own consent. Therefore, among the patients that satisfied the criteria described above, 5 patients whom spouse, adult child, parent or adult sib provided consent were used as test subjects in the inhalation of hydrogen-supplemented oxygen. Table 2 shows the details of the 5 test subjects.

TABLE 2

| | | | | Putative | |
|---|---|---|---|---|---|
| | | | Initial | cardiac arrest | |
| Patient | Age | Sex | waveform | duration (min) | Diagnosis |
| 1 | 65 | Female | Asystole | 39 | Pneumonia |
| 2 | 77 | Male | VF | 15 | Hyperkalemia |
| 3 | 47 | Male | VF | 16 | AMI |
| 4 | 80 | Female | VF | 14 | HOCM |
| 5 | 57 | Male | VF | 24 | AMI |

HOCM: hypertrophic obstructive cardiomyopathy
AMI: acute myocardial infarction

2. Test Drug

In this test, the hydrogen gas inhalation of the test subjects was performed using hydrogen-mixed nitrogen (4% hydrogen and 96% nitrogen) contained on the inspiration side of an existing mechanical ventilator, together with oxygen (i.e., the composition of the hydrogen-supplemented oxygen was as follows: H₂: 2%, O₂: 50%, N₂: 48%). There is no previous report on hydrogen gas inhalation in humans. For sufficient consideration given to its safety, the present inventors tested this time the safety of a hydrogen gas administration method from the side tube of the mechanical ventilator, and necessary amounts of the gases supplied, and confirmed that this test was executable safely for humans.

3. Administration

By consent of test subject's spouse, adult child, parent or adult sib, the administration of the hydrogen-supplemented oxygen was started as quickly as possible under temperature control treatment after entrance into a general intensive care unit, and continued to 18 hours later under a doctor's monitoring.

The time from the return of spontaneous circulation to the hydrogen-supplemented oxygen was as shown in Table 3 below.

TABLE 3

| | Time from return of |
|---|---|
| | spontaneous circulation to |
| Patient | start of administration |
| 1 | 3 hours and 41 minutes |
| 2 | 7 hours and 2 minutes |
| 3 | 5 hours and 23 minutes |
| 4 | 4 hours and 56 minutes |
| 5 | 3 hours and 47 minutes |

4. Evaluation Item

The efficacy of the hydrogen gas inhalation was evaluated on the basis of survival at day 90 of disease and the ratio of good cerebral performance (CPC1) conforming to the Glasgow-Pittsburg Cerebral Performance Category (CPC).

5. Results

Table 4 shows the survival rate and CPC1 ratio of the test subjects (hydrogen inhalation group) at day 90 of disease. Table 5 shows the average ages of the hydrogen inhalation group and a control. The control refers to patients who did not inhale the hydrogen-supplemented oxygen among the patients selected according to the paragraph 1.

TABLE 4

| | | Hydrogen | |
|---|---|---|---|
| | Control | inhalation | |
| | N = 6(%) | N = 5(%) | P value |
| 90 Days Survival | 5(83) | 4(80) | 0.73 |
| 90 Days CPC 1 | 3(50) | 4(80) | 0.35 |

TABLE 5

| | | Hydrogen | |
|---|---|---|---|
| | Control | inhalation | |
| | N = 6(%) | N = 5(%) | P value |
| Age, y/o* | 43(13) | 65(14) | 0.03 |

*mean (SD)

According to the report released by the Fire and Disaster Management Agency of Japan on the survival rate, etc. of patients who suffer cardiopulmonary arrest, the 1-month survival rate of survivors of cardiogenic arrest of cardiopulmonary function tends to decrease with age irrespective of sex, and significantly decreases in 70-year-old or older patients (http://www.fdma.go.jp/neuter/topics/houdou/h21/2101/210122-1houdou_h.pdf, Attachment 1-2). Likewise, according to the report, the rehabilitation rate after 1 month of the survivors of cardiogenic arrest of cardiopulmonary function tends to decrease with age irrespective of sex, and significantly decreases in 60-year-old or older patients.

The survival rate of the patients at day 90 of disease was 83% for the control who did not inhale hydrogen, whereas the survival rate was 80% for the hydrogen inhalation group. Thus, almost equal outcomes were obtained. As for the ratio of CPC1, the hydrogen inhalation group exhibited a higher percentage (80% vs 50%). The average age of the patients was higher by 20 years or more in the hydrogen inhalation group than in the control, and the average age of the hydrogen inhalation group was 65 years old. In light of these facts, the results obtained about the improvement of survival rates and brain function by the hydrogen inhalation indicate the significant effect of the hydrogen inhalation.

More surprisingly, the outcomes described above were brought about by the hydrogen inhalation performed after a lapse of at least 3 hours from the return of spontaneous circulation in the test subjects. As already mentioned, reactive oxygen species responsible for multiple organ damage are considered to be generated several minutes after return of spontaneous circulation. Therefore, in such a case where a considerable period of time has passed after return of spontaneous circulation, multiple organ damage ascribable to reactive oxygen species has already progressed. Hence, it is thought that the effect brought about by the removal of reactive oxygen species by hydrogen gas cannot be expected. Thus, the outcomes described above cannot be explained by current technical common sense and are significant, suggesting that there exists another action different from the removal of reactive oxygen species by hydrogen gas.

### INDUSTRIAL APPLICABILITY

The pharmaceutical composition of the present invention can improve prognosis after return of spontaneous circulation even after a lapse of a considerable period of time from the return of spontaneous circulation.

1. A method for improving prognosis after return of spontaneous circulation in a human subject after a lapse of 30 minutes or longer from the return of spontaneous circulation following cardiopulmonary arrest for at least 10 minutes, the method comprising administering to the human subject a gaseous pharmaceutical composition comprising hydrogen gas,
wherein the prognosis after return of spontaneous circulation is improved in a human patient with the administration of the pharmaceutical composition as compared with a human patient group without the administration of the pharmaceutical composition.

2. The method according to claim 1, wherein the improvement of the prognosis is improvement of brain function.

3. The method according to claim 1, wherein the human subject is a subject having a Glasgow Coma Scale (GCS) score of 8 or lower.

4. The method according to claim 1, wherein the improvement of prognosis after return of spontaneous circulation is improvement at at least 90 days after the return of spontaneous circulation.

5. The method according to claim 1, wherein the improvement of prognosis after return of spontaneous circulation is determined on the basis of a Cerebral Performance Category (CPC) score.

6. The method according to claim 1, wherein the pharmaceutical composition is continuously administered for at least 18 hours from the start of administration.

7. The method according to claim 1, wherein the pharmaceutical composition farther comprises oxygen gas.

8. The method according to claim 1, wherein the pharmaceutical composition further comprises an inert gas.

9. The method according to claim 1, wherein the hydrogen concentration in the pharmaceutical composition is 0.1% to 4.0%.

10. The method according to claim 9, wherein the hydrogen concentration in the pharmaceutical composition is 1.0% to 2.0%.

11. The method according to claim 1, wherein the cardiopulmonary arrest is caused by acute myocardial infarction, cardiomyopathy or hyperkalemia.

12. The method according to claim 1, wherein the hydrogen gas is provided by a hydrogen gas generation apparatus or a container containing the hydrogen gas.

13. The method according to claim 12, wherein the hydrogen gas generation apparatus generates hydrogen generation by water electrolysis.

14. The method according to claim 12, wherein the container contains a mixed gas of the hydrogen gas and nitrogen gas.

15. The method according to claim 12, wherein the hydrogen gas generation apparatus or the container comprises a device for gas inhalation, together with piping connected to the apparatus or the container and an inhalation means for gas inhalation connected to the piping.

16. The method according to claim 15, wherein the device for gas inhalation further comprises an oxygen gas generation apparatus or a container containing oxygen gas.

17. The method according to claim 15, wherein the device for gas inhalation further comprises a means which controls a gas flow rate to the inhalation means.
